(19) United States
(12) Patent Application Publication
(10) Pub. No.: US 2012/0172388 A1
(43) Pub. Date: Jul. 5, 2012

(54) Enhancement of Impaired Motor and Mental Functions, Using Dextromethorphan and Oxidase Enzyme Inhibitor

(76) Inventor: Richard A. SMITH, La Jolla, CA (US)

(21) Appl. No.: 13/415,990

(22) Filed: Mar. 9, 2012

Related U.S. Application Data
(63) Continuation of application No. 13/191,911, filed on Jul. 27, 2011.
Application No. 13/191,911 is a continuation of application No. 11/246,883, filed on Oct. 7, 2005, now abandoned.
(60) Provisional application No. 60/616,884, filed on Oct. 7, 2004.

(51) Int. Cl. A61K 31/485; A61P 25/16

(52) U.S. Cl. 514/289

(57) ABSTRACT

During clinical trials on patients suffering from neurological disorders, it has been observed that some patients obtain dramatic improvements in motor control and/or higher mental functioning, when they receive a combination of dextromethorphan and quinidine, at suitable dosages. Improved motor control has been exemplified to date by improved ability to swallow and/or speak, among victims of stroke, head injury, or ALS. Improved higher mental functioning has been exemplified in better job performance, increased ability to analyze and solve problems, and increased ability to have successful and satisfying interactions with other people. These types of effects can be seen in a relatively brief time period, such as within several days to a week.

### RELATED APPLICATION

This invention claims the benefit under 35 USC 119 of provisional application 60/616,884, filed on Oct. 7, 2004.

### FIELD OF THE INVENTION

This invention is in the field of pharmacology, and relates to a combination of compounds that, when taken together, have led to enhanced motor control and mental performance, in patients who suffer from neurological impairments.

### BACKGROUND OF THE INVENTION

Dextromethorphan is the common name for (+)-3-methoxy-N-methylmorphinan. It is a non-addictive opioid that has a “mirror image” of the morphinan ring structure, which forms the molecular core of most opiates. It is widely used as cough suppressant, and is described in numerous articles and in any recent edition of Goodman and Gilman's Pharmacological Basis of Therapeutics.

Quinidine is a well-known compound that has been used for many years to treat certain types of cardiac arrhythmias. For unrelated reasons, it also can inhibit a certain enzyme (present mainly in the liver) that oxidizes and degrades dextromethorphan. That enzyme, which belongs to a family of enzymes known as “cytochrome P450” enzymes, initially was called debrisoquin hydroxylase, and sparteine monooxygenase. The most widely-used name today is the P450-2D6 enzyme.

Dextromethorphan (abbreviated herein as DM) is known to have relatively weak activity at an important class of neuronal receptors inside the central nervous system (the CNS, which includes the brain and spinal cord). Those receptors are called NMDA receptors, and they are triggered by glutamate, one of the two major excitatory neurotransmitters in mammalian nervous systems (the other is acetylcholine). Because of the roles and importance of glutamate and NMDA receptors in the mammalian CNS, DM began to be tested, in the late 1980's and early 1990's, in patients suffering from various neurological disorders (such as amyotrophic lateral sclerosis, ALS, also called Lou Gehrig's disease), to see if it might be able to help slow the progression of any of those diseases. Regrettably, it did not show enough benefits in those tests to justify larger trials.

However, during the course of testing DM on patients suffering from such disorders, the Applicant herein recognized that much of the variability in the effects of DM on different patients apparently arose because patients were metabolizing and eliminating DM from their blood, at widely differing rates. After he began looking into that factor in detail, he identified the P450-2D6 enzyme as the most important DM-degrading enzyme, and he located two published reports (Inaba et al 1985 and 1986) indicating that various known drugs could inhibit the P450-2D6 enzyme with varying levels of potency. The most potent P450-2D6 inhibitor identified in those reports was quinidine.

Accordingly, the Applicant began testing quinidine as an adjunct for use with DM, to protect the DM against rapid metabolic degradation in patients being tested.

Soon after those tests began, the Applicant began to notice and realize that the combination of DM-plus-quinidine was creating unexpected but highly valuable and useful results, not in all patients, but in some patients who were receiving the combination.

One of the first such observations was that some patients who being tested for other neurological disorders were obtaining unexpected but effective relief from a condition known by several names, including pseudobulbar affect, and emotional lability. Victims of neurological diseases, strokes, or head injuries who suffer from this problem lose the ability. to control their emotions and emotional displays, and may begin to laugh loudly, or weep uncontrollably, at inappropriate moments and with no apparent reason. This disorder can become very disturbing and deeply distressing to a person who is affected by it, and to friends and family. It often drives people who suffer from this condition to become hermits and recluses, afraid to go to restaurants, theaters, or other public places, out of fear that they will humiliate themselves (and anyone who is with them) by suddenly launching into inappropriate, unwelcome, and disruptive emotional displays.

When the Applicant began testing DM/quinidine combinations on patients who happened to suffer from pseudobulbar problems (as secondary problems that accompanied a primary neurological problem that led to their enrollment in a clinical trial), several of those patients began to report major relief from their emotional lability problems. They began to report that they were much more capable of controlling their emotions and emotional displays, and they had become more capable of maintaining an “even keel” in dealing with life's daily events, and in interactions with family and friends.

As a result, the Applicant filed two patent applications, which issued as U.S. Pat. No. 5,166,207 (Smith 1992) and U.S. Pat. No. 5,206,246 (Smith 1993). Those early results were also described in Zhang et al 1992, coauthored by the Applicant. Those discoveries were later followed by discoveries of additional medical uses for the DM/quinidine combination, as described in U.S. Pat. No. 5,366,980 (on treating severe dermatitis), U.S. Pat. No. 5,350,756 (increasing the cough-suppressing efficacy of DM), reissue 38,115 (arising from U.S. Pat. No. 5,863,927, on treating chronic pain and certain other disorders), and U.S. Pat. No. 6,207,674 (weaning patients from narcotics, anti-depressants, and certain other drugs). The contents and teachings of those patents are incorporated herein by reference, as though fully set forth herein.

The DM/quinidine combination has been licensed to a pharmaceutical company called Avanir (LaJolla, Calif.; www.avanir.com), which is sponsoring several clinical trials on the combination. Those trials are at various stages of planning or progress, as can be monitored from postings on the Avanir website, or from other websites that track clinical trials that have received partial approvals to proceed, from the U.S. Food and Drug Administration. Phase 3 results for treating pseudobulbar affect (emotional lability) were announced by Avanir in August 2004. The results reportedly were excellent, leading Avanir to file a New Drug Application on the DM/quinidine combination (which has been given the trademark NEURODEX). As this is being written, in October 2005, Avanir hopes and expects to receive full approval to sell the drug combination to patients who need it, on a prescription basis (it should be noted that quinidine, originally developed as a heart medicine, can be dangerous if taken by people who suffer from a heart condition known as a “prolonged QT interval”; therefore, patients who are being evaluated to determine whether they should receive it should first be given an electrocardiogram, to ensure that they do not suffer from that particular heart condition).

This current application discloses yet another apparent and potential medical use for the DM-plus-quinidine combination. This new apparent use has been observed recently by the Applicant, during the course of clinical studies that were being carried out to evaluate the efficacy of the DM/quinidine combination for other medical needs (also called “indications” in FDA terminology). Not all patients suffer from these problems, and it is not asserted herein that all patients will benefit from this treatment, in the manner disclosed herein. Instead, the discovery and invention herein centers on and arises from the fact that this treatment can and will help a subcategory of patients, and patients who can and will be helped by this treatment can be identified fairly easily, and tested by means of routine screening and evaluation.

To adequately describe and explain the effects that have been recently observed, additional background information needs to be addressed, under the next two subheadings.

### Motor Skills, and Mental Skills

During a series of clinical trials that were focusing mainly on other problems, the Applicant began to observe (and patients began to report) substantial improvements in other areas that had been causing trouble for certain patients. Those areas can be broadly and generally classified as (1) motor skills, and (2) mental skills. Those areas are not entirely separate and distinct, and they can overlap with each other in some patients.

In general, motor skills include activities that require coordinated actions involving both nerves and muscles. These can be exemplified by activities such as swallowing, speech, walking, use of the hands etc.

In particular, speech and/or swallowing are often impaired in patients who have suffered a stroke, head injury, neurodegenerative disease, or other problem. Speech impairments are noticed and monitored by family members, caregivers, and others who must communicate with the patient, and who often describe the patient's speech as slurred, garbled, unclear, etc. Swallowing is also a frequent problem, which affects not just the ability to eat and drink, but also the ability to cope with saliva production; if a patient cannot swallow his or her saliva easily and regularly, it can lead to serious medical problems, including fluid accumulation in the lungs, which can lead to pneumonia and other life-threatening infections.

A number of patients with speaking or swallowing problems were enrolled in clinical trials of DM/quinidine for treating other neurological conditions. Surprisingly, a number of those patients enjoyed major improvements in their ability to speak and/or swallow. Several examples are described below.

Other patients showed substantial and even major improvements in mental skills that do not require accompanying or coordinated actions involving muscles or motor control. These types of mental skills and functions (which are sometimes referred to as “higher” mental skills) include cognition, reasoning, memory, etc. These types of effects and results are not as easy to describe or classify as improvements in motor skills, such as swallowing or speaking; however, major and even life-changing improvements in “higher” mental functioning have been observed in several patients who initially began taking the DM/quinidine combination for other reasons. An adequate explanation of these types of effects requires some additional information, under the next subheading.

### Excitatory and Inhibitory Systems in the Brain

To help readers who are not experts in neurology understand certain aspects of this invention, an analogy is used herein, which compares coherent thinking, in a brain, to a coherent picture on a television set. As can be readily understood, a properly-working television must perform two different functions. The first function is obvious: the television must be able to receive, process, and display images from a particular channel, at a particular moment in time.

The second function is less obvious and often goes unrecognized, but it is equally important. A television set also must be able to filter out, suppress, and not display all of the other, competing signals that are being sent to it, by the channels that are not being watched at some moment in time. If the conflicting and competing images that cannot be filtered out and suppressed, the image on the screen will be an unpleasant jumble of unsorted, incoherent images.

If either the receiving or the filter-and-suppress function is partially impaired, the result on a television usually appears as static, “ghost” images, shadow images, crosstalk, failure of horizontal or vertical control to maintain a stable image, etc. Those problems can range from mildly annoying, to a point that renders a television set worthless and unusable.

For similar reasons, both excitatory and inhibitory transmitter and receptor systems must work together, in a coordinated manner, in the brain. As indicated by the name, when an excitatory neurotransmitter is released by a neuron into a synaptic junction (i.e., the fluid-filled gap between a transmitting neuron and a receiving neuron), the excitatory transmitter normally will trigger a “firing event” (also called a depolarization, nerve impulse, nerve signal, etc.) in the receiving neuron. One or more “ion channels” in the outer membrane of the receiving neuron will open, and for a few milliseconds, positive and negative charged ions will rush through the open channel, into and out of the neuron, in a way that decreases a voltage gradient across the membrane, from a “resting state” that typically is about 90 millivolts in most types of neurons, to a “depolarized” voltage of about 65 millivolts. This drop in the voltage gradient across the neuron's outer membrane triggers various “downstream” events, which collectively comprise a “firing” event for the neuron. The ion channel that is controlled by the synaptic receptor will rapidly close, and the neuron will turn on various “ion pumps” that will begin pumping ions into and out of the neuron, until it reaches its desired high-voltage resting state, which will render it ready to receive the next nerve impulse.

However, excitatory transmitters and receptors are only half of a complete set, and inhibitory transmitters and receptors provide the other half. As indicated by their name, inhibitory transmitters and receptors regulate and filter out unwanted nerve signals, which otherwise would lead to problems that would be analogous to static, ghost images, shadows, and crosstalk on a television that is not working properly.

One way to mentally grasp the differences between excitatory versus inhibitory receptors is to recognize that most excitatory receptors are positioned at the tips of the fibers that extend out from a neuron. Since a typical neuron has dozens or even hundreds of such fibers, these receptors allow a neuron to communicate with dozens or hundreds of other neurons. By contrast, most inhibitory receptors tend to be positioned somewhere along the length of a nerve fiber, where they can function as gates, or valves, that will control the flow of liquids through the “pipe” provided by the nerve fiber.

The two main excitatory neurotransmitters are glutamate, and acetylcholine. Either of those molecules can trigger an impulse or “firing” event, in a signal-receiving neuron. However, both of those two excitatory transmitters can interact with numerous different types of neuronal receptors. Glutamate interacts with three different types of glutamate receptors, which were named after artificial probe drugs that are not used in nature, but that can bind selectively to those three subclasses of glutamate receptors, under laboratory conditions. Those three types of glutamate receptors are called NMDA receptors, kainate receptors, and AMPA receptors. Similarly, acetylcholine can trigger either muscarinic and nicotinic receptors, both of which are subdivided into still more subtypes.

In addition to glutamate and acetylcholine (the two most important excitatory transmitters), several other lesser-but-crucial excitatory transmitters and/or receptors are known. Neuropeptide Y, a protein, is an excitatory neurotransmitter, but it is not entirely clear which receptors are bound and activated by it. Sigma receptors also are known to be excitatory, but it is not entirely clear which neurotransmitters trigger their activity; since dextromethorphan has some level of activity at sigma receptors, they are discussed in more detail below. Epinephrine (also called adrenaline) and norepinephrine can also act as neurotransmitters inside a mammalian brain; however, they do not have the same effects in the brain as in the rest of the body, and curiously, both molecules apparently can play either excitatory or inhibitory roles inside a brain, depending on which portions of the brain are involved.

Inhibitory neurotransmitters also involve numerous different transmitters and receptors. Dopamine and serotonin are modulating agents, which are heavily involved in the “pleasure centers” of the brain. Gamma-amino-butyric acid (GABA) usually acts in a manner comparable to an on-off switch, making “direct” GABA agonists useful for surgical anesthesia, to render a patient or limb totally insensitive to pain; however, “indirect” GABA agonists (such as benzodiazepine drugs, including VALIUM™) have been developed that have only indirect activity at GABA receptors (they slightly increase the levels of GABA in blood or cerebrospinal fluid), and such drugs can act as anxiolytics and sedatives. There are also various “opiate” receptors (also called opioid receptors), which can be activated by natural endorphins, but which can be triggered more powerfully by synthetic drugs such as morphine. Still other receptors that appear to be mainly inhibitory have been identified because they interact with certain known drugs; this includes “cannabinoid” receptors, which apparently are triggered by the active agents in marijuana.

Dextromethorphan (DM) has a complex combination of activities. It mildly suppresses activity at NMDA receptors, which are excitatory, but it also stimulates activity at sigma receptors, which also are excitatory.

In addition, a few reports in the 1980's indicated that DM also binds to both “high-affinity dextromethorphan receptors” and “low-affinity dextromethorphan receptors” (e.g., Craviso et al 1983, Musacchio et al 1988a and 1988b). However, relatively little has been published on those putative receptors since then, and a 1992 report (Klein et al 1992) contained data suggesting that the “high-affinity” DM receptor may actually be the sigma-1 receptor, while other reports (e.g., Franklin et al 1992 and Church et al 1994) contained data suggesting that the “low-affinity” DM receptor may actually be a part of the NMDA receptor and ion channel complex. It should be noted that during the late 1980's and early 1990's, major advances were being made in identifying and studying numerous different types of neuronal receptor and transmitter system, and in studying and recognizing both differences and similarities between receptor types in very different species (such as mice and humans). Therefore, it was not uncommon when skilled researchers, carrying out complex research from different angles and starting points, converged at what were later recognized as common or at least shared meeting points.

To make matters even more complex, it must be recognized that neuronal receptors (either excitatory or inhibitory) respond in totally different ways when triggered by “agonist” or “antagonist” molecules. Agonists are molecules that will trigger or otherwise promote or increase the “natural” response in a certain type of receptor (bearing in mind that a “natural” response might involve either boosting or suppressing the transmission of nerve impulses, depending on whether a receptor is an excitatory or inhibitory receptor). By contrast, antagonist molecules that tend to block and suppress the “natural” response of a particular receptor type (most commonly, by occupying a receptor in a “competitive binding” manner, which will prevent the normal and natural triggering agents from reaching and activating the receptor).

However, even the identification and characterization of agonists or antagonists can be complex, and can involve shades of gray. For example, a drug molecule that binds to a certain receptor might initially act as an agonist, by initially triggering the natural response by that particular receptor. However, if that drug clings to the receptor and occupies it for an abnormally long time (thereby preventing the receptor from being “reset”, and thereby inhibiting its ability to participate in subsequent activation events), the drug can act as an antagonist, by suppressing the receptor's activity. Because of these and other factors, many articles refer to certain drugs as “ligands” of various receptors (the term “ligand” indicates that a certain molecule binds to a certain type of receptor, without indicating or implying whether the ligand has either agonist or antagonist activity).

It also must be recognized that the systems and networks of neurons and synapses, in a human brain, are constantly changing, and are not static. A thought, memory, or other mental construct or connection is not contained in single neuron; instead, thoughts and memories are created and preserved by the ways neurons are connected to each other, in clusters and networks that are controlled by the strengths and activity levels of millions or billions of synaptic junctions between neurons. Every day, new synaptic connections are being made, as people experience and remember new things, while other synaptic junctions are being weakened or disconnected, as people forget trivialities, things they have not thought of for weeks or years, etc.

In addition, anyone interested in neurology should also recognize that the organization of a mammalian brain is extraordinary. Even in a mammal as small as a mouse, hundreds of distinct regions and specialized structures must interact with each other in carefully controlled ways. This includes, in particular, numerous structures that must govern the flow, handling, and prioritizing of signals between various portions of the brain, in ways that are analogous to a central switchboard in a major telephone center, or a control room where operators monitor and govern all of the components of a major refinery, nuclear power plant, or busy airport.

If any of these systems malfunctions, the brain can malfunction, in ways that are analogous to static, ghost or shadow images, or a loss of horizontal or vertical control, in a television set. The results can range anywhere from barely noticeable, at one end of the scale, to overwhelming psychoses, at the other end of the scale.

Unless a patient is suffering from a known small and localized tumor, lesion, or injury, it is effectively impossible to know which neurons, synapses, or networks are causing or aggravating a problem in motor control or mental functioning. However, that level of knowledge is not required, in order to recognize, understand, and effectively utilize the effects that a DM/quinidine combination can have, on patients who will respond to such treatment in a desired and useful manner. As described below, a practical treatment, using a known, non-toxic, well-tolerated drug combination, has been discovered that has enabled numerous patients who were suffering from various different serious or severe neurologic impairments to “adjust the tuning” in their brains.

Continuing the television analogy from above, a typical homeowner who uses an outdoor antenna for his television knows enough to be able to rotate the antenna, until the picture being carried by a certain channel on his television reaches a “best available” level. That is a practical solution, which can be carried out even if a homeowner has no idea how electronic circuits, electromagnetic signals, or tuning electronics actually work.

In an analogous manner, in clinical trials involving people who were suffering from various types of serious neurologic impairments, when dextromethorphan was accompanied by an oxidase enzyme inhibitor that helped sustain higher concentrations of the DM, in circulating blood, a practical and enormously useful discovery was made. In some of those patients, those two combined drugs were discovered to be extraordinarily effective and useful in helping those patients improve their motor control and/or mental functioning. It helped quiet down and control the types of static, distractions, and unwanted noise that had been interfering with their ability to focus on, process, and utilize clear signals and thoughts. These results are described in more detail, below.

Accordingly, one object of this invention is to disclose that a DM-plus-quinidine combination (or other drug combinations that can interact with multiple neuronal receptor types in similar ways) can provide major improvements in motor control, such as swallowing and speaking, among some patients who suffer from impaired motor control.

Another object of this invention is to disclose that a DM-plus-quinidine combination (or other drug combinations that can interact with multiple neuronal receptor types in similar ways) can provide major improvements in various types of higher mental functioning, including improved cognitive, analytical, communicative, memory, and others skills that can improve job performance, interpersonal relationships, or other activities, among some patients who suffer from impaired higher mental functioning.

Another object of this invention is to disclose a new method for treating patients with neurological problems who suffer from impaired motor control or mental functioning.

Another object of this invention is to disclose a new method for treating patients with neurological problems who suffer from cognitive, reasoning, and/or memory disorders or impairments, to help such patients reach and sustain improved levels of cognitive, reasoning, memory, or other mental functioning and performance.

Another object of this invention is to disclose a method for screening patients who suffer from problems involving motor control or mental skills, to identify which such patients will benefit from a treatment regimen that includes a DM-plus-quinidine (or similar) combination.

These and other objects of the invention will become more apparent through the following summary and description.

### SUMMARY OF THE INVENTION

During clinical trials involving patients who suffer from various types of neurological disorders, it has been observed that some patients obtain dramatic improvements in motor control and/or higher mental functioning, when they receive a combination of dextromethorphan and quinidine, at suitable dosages.

Improvements in motor control skills have been exemplified to date by improved ability to swallow and/or speak, among people such as victims of stroke, head injury, or ALS. Screening tests are described herein to determine whether these treatments will also be able to provide substantial benefits in other types of motor control, in various types of patients (such as, for example, improved ability to walk, among some patients with cerebral palsy, and improved hand stability, among some patients who suffer from Parkinson's disease or other disorders that cause trembling, spasms, etc.).

Improvements in higher mental functioning have been exemplified to date by better job performance, increased ability to analyze and solve problems, and increased ability to have successful and satisfying interactions with other people. In a number of cases, patients who previously were living on the outer edges of society and functionality, due to mental disorders or impairments that rendered them unable to cope adequately with the demands of daily life (including, in some cases, impairments created by severe traumatic head injuries) have reported dramatic and life-changing improvements in their ability to filter out mental distractions, focus on what is important, make better decisions based on improved cognitive and reasoning abilities, all of which have led to major strides forward in their lives and careers.

These types of effects can be seen in a relatively brief time period, such as within several days to a week. Accordingly, screening methods are disclosed, which can be used to identify and begin helping patients who will benefit from such treatment.

### BRIEF DESCRIPTION OF THE DRAWINGS

FIG. 1 depicts a dextromethorphan analog with a fluorine atom substituted for a hydrogen, to enable researchers to use various types of imaging methods to learn more about dextromethorphan binding sites in mammalian brains.

### DETAILED DESCRIPTION

As summarized above, new and recent observations by the Applicant have indicated that, in some patients who have received a DM-plus-quinidine combination during clinical trials involving various neurological impairments, the drug combination has provided major improvements in various types of motor control, exemplified by swallowing and speaking, which are common motor control problems among patients with the types of neurological disorders that would lead to inclusion in such trials.

In addition, in some patients involved in those trials, the DM/quinidine combination has led to substantial and even life-transforming improvements in mental functioning, including cognitive, reasoning, and memory skills.

For convenience, these two different but potentially overlapping types of changes are classified and referred to herein as motor improvements, and mental improvements. Three examples of improved motor control and performance are described in Examples 1-3, and three examples of improved mental functioning and performance are described in Examples 4-6.

Suitable dosages will vary among different patients, depending on factors such as their size, age, weight, gender, and metabolic rates. Since quinidine can be administered by prescription only (due to the risks it can pose in patients who have prolonged QT intervals in their heartbeat), preferred dosages can be determined with monitoring and supervision by a qualified physician. To ensure tolerability, most adults preferably should commence with a testing period, such as 3 to 7 days, using a relatively low dosage (such as about 10 to about 30 mg/day) of DM only, with no quinidine. If no adverse effects are encountered, a second brief trial can use a combination of, for example, 10 mg DM and 10 mg quinidine per day. If no adverse effects are encountered, a dosage combination of about 25 to 30 mg/day of each drug can provide a convenient testing level for most patients. Either or both of those two dosage levels can be increased or decreased if desired, under the supervision of a physician. Unless otherwise determined by an experienced physician for a specific patient, dosages generally should be kept to less than about 100 mg/day of each drug.

It is not claimed or asserted that a DM/quinidine combination can treat all such problems, or all patients who suffer from such problems. Instead, the current state of understanding and belief can be summarized as follows:

(1) The DM/quinidine combination can and will help at least some patients who suffer from motor control impairments that accompany and/or are caused or aggravated by various neurological disorders. The range and variety of the types of neurological disorders and/or motor control impairments that may be helped by the drug combination have not yet been extensively evaluated, and are not yet fully known. However, major benefits have been seen in patients suffering from totally different types of neurological impairments, including: (1) amyotrophic lateral sclerosis (ALS, also called Lou Gehrig's disease), a slow and progressive neurodegenerative disease; (2) multiple sclerosis, a slowly-progressing disorder that has skeleto-muscular as well as neurological components; and, (3) traumatic head injury. Since patients suffering from such a wide range of disorders have showed major benefits from this treatment, then a working presumption arises, as follows. If a neurological disease or disorder causes motor control problems (such as difficulty in swallowing, slurred or garbled speech, etc.) in a specific patient, then such patient offers a good candidate for testing and screening, to determine whether the DM/quinidine combination will benefit that particular patient, unless the patient has a heart condition known as a “prolonged QT interval”.

(2) People who suffer from motor control problems involving one or more limbs (such as leg or arm weakness, impaired ability to walk, tremors or trembling in the hands, spasticity, etc.), related to factors such as stroke damage, cerebral palsy, Parkinson's disease, or other disorders, also can be evaluated using a DM-plus-oxidase-inhibitor combination, and it is currently believed that at least some such patients are likely to receive at least some benefits from this treatment.

(3) Patients for whom the DM/quinidine is contraindicated include two categories of patients.

First, patients with a heartbeat condition known as a “prolonged QT interval” (this condition is well-known to physicians, and can be detected easily by an electrocardiogram) should not take quinidine, which can aggravate that irregularity. However, there are other known drugs that can inhibit the cytochrome P450-2D6 oxidase enzyme, the major enzyme that degrades dextromethorphan; a number of such drugs are listed in Inaba et al 1985 and 1986, and still others have been discovered since then. Therefore, patients who have a prolonged QT interval can take one of those alternate P450-2D6 oxidase inhibitor drugs, as a substitute for quinidine, along with dextromethorphan.

Second, some patients who are taking various other drugs (as is quite common among nearly all patients who suffer from substantial neurological disorders), or who have certain types of enzyme profiles, may suffer hallucinations, if given a DM/quinidine combination at dosages that typically involve 50 or 60 mg/day of DM, and 50 or 60 mg/day of quinidine. Accordingly, any candidate patient should be initially tested for DM/quinidine tolerance and side effects, using a relatively low dosage of either or both drugs (such as 25 to 30 mg/day), under controlled and non-dangerous conditions that will allow the patient to be calmed and reassured if such side effects begin to arise.

Those two caveats were both recognized and accounted for at an early stage of the research. Since then, the DM/quinidine combination has been well-tolerated by most patients tested, and any side effects that occur are relatively benign and non-severe, and they dissipate and cease fairly rapidly, after a person stops taking the drug combination.

(4) The onset of any symptomatic changes is fairly rapid, and usually becomes apparent within a day or two, or possibly a week at most. This is in contrast to various types of drugs that often take several weeks or even months before they begin exhibiting effects that are noticeable to the user.

In view of the excellent tolerability, minimal side effects, and rapid onset of noticeable changes caused by the DM/quinidine combination, it is a simple and straightforward matter for any candidate patient who suffers from a motor control problem to simply try the drug combination, to determine whether it will provide substantially improved motor control for that particular patient. Accordingly, this approach offers a useful, effective, and relatively rapid screening option. While clinical trials can and should be done to gather statistical data on the types and ranges of motor control problems and conditions that can be helped, such trials do not need to be completed and thoroughly evaluated, before patients and their physicians can simply try this combination on a trial basis, using a low-dosage tolerability test at the start of the test, to find out whether it will help a specific patient who suffers from a specific type of motor control problem.

Neuronal Receptor Types that are Involved

As mentioned in the Background section, dextromethorphan (DM) is known to act at at least two, possibly three, and possibly four different types of neuronal receptors, in a human brain. All of those receptor types require some attention, because the discovery herein also suggests that various combinations of other drugs that can exert the same types of effects at the same types of receptors may be able to accomplish similar or possibly even improved results.

First, DM is known to suppress activity at the NMDA class of glutamate receptors. These receptors normally are activated by glutamate, the most important excitatory neurotransmitter in mammalian brains. NMDA receptors have been studied very extensively, and they are described in numerous review articles, such as Waxman et al 2005 and Perez-Otano et al 2005.

Second, DM is also known to stimulate activity at sigma receptors, or at least at the sigma-1 subclass of receptors. Sigma receptors are not understood nearly as well as NMDA receptors. Under natural conditions, they are believed to respond mainly to certain types of “neurosteroids”, which are neurologically-active compounds that are synthesized from the same starting molecular structures as steroids, in the remainder of the body, and they are believed to perform a variety of different roles, including neuroprotective activity, intracellular amplification of certain types of signals, enhancement of memory formation, and preventing both diarrhea, and depression. In addition to DM, a number of sigma agonists are known, including drugs that are identified by numbers such as JO1783 (also known as igmesine), OPC-14 523, and SA4503. Various other drugs (such as opipramol and siramisine) that function as sigma ligands, and that reportedly have at least some level of sigma agonist activity, are discussed in articles such as Volk et al 2004. Still other drugs that are believed to function as sigma antagonists (blockers) are known, including rimcazole and progesterone. Review articles that describe sigma receptors include Maurice et al 1997 and 2002, Baulieu 1998, Su et al 2003, Maurice 2004, Skuza et al 2004, Takebayashi et at 2004, and Guitart et al 2004, and most of these articles also describe the effects of various known sigma receptor ligands.

In addition, as noted in the Background section, a few reports in the 1980's indicated that DM also binds to “high-affinity dextromethorphan receptors” and “low-affinity dextromethorphan receptors” (e.g., Craviso et al 1983, Musacchio et at 1988a and 1988b). However, subsequent reports appeared to suggest that “high-affinity” DM receptors may actually be sigma-1 receptors (e.g., Klein et al 1992), while other reports appeared to suggest that “low-affinity” DM receptors may actually be part of the NMDA receptor and ion channel complex (e.g., Franklin et al 1992 and Church et at 1994). Therefore, it is not known with certainty whether separate classes of either high-affinity or low-affinity DM receptors even exist, in various types of animal species such as mice or rats, or in humans or other primates.

Those various unknown factors are likely to become of substantially greater interest, among neurology researchers, after they become aware of the discoveries described herein, and the dramatic nature of the effects that a DM/quinidine combination has created, in patients who are suffering from serious neurological impairments.

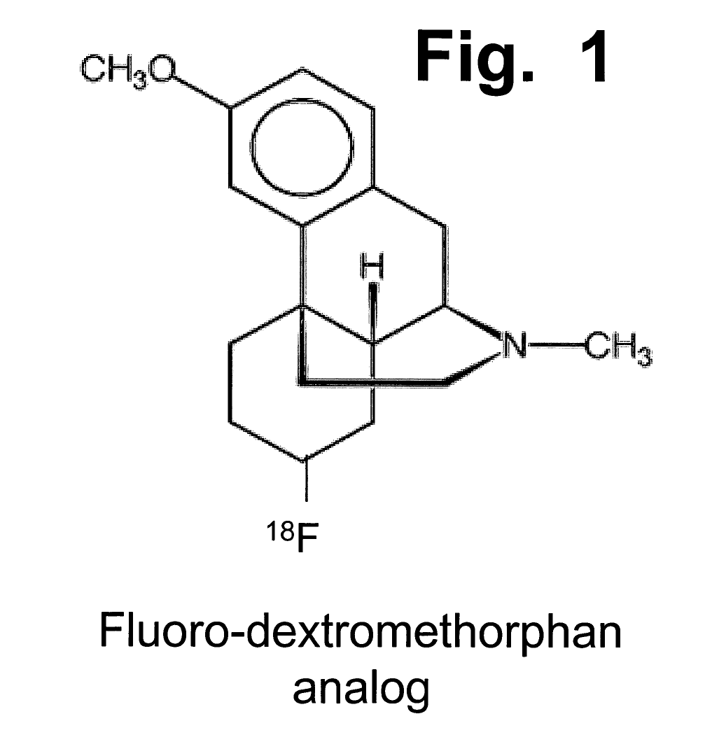

Accordingly, in an effort to facilitate and accelerate those efforts, the Applicant is disclosing herein a new analog of dextromethorphan, which contains a fluorine atom at a selected location in the molecule, illustrated in FIG. 1. That particular location in the molecule will enable convenient synthesis of that fluorine analog, using known methods; however, synthetic chemists will also recognize other candidate substitution sites as well. Various methods of synthesizing DM and its analogs are described in U.S. Pat. No. 3,914,233 (Mohacsi et al 1975), U.S. Pat. No. 4,388,463 (Brossi et al 1983), U.S. Pat. No. 4,390,699 (Brossi et al 1983), and U.S. Pat. No. 4,552,962 (Brossi 1985), and in various earlier articles and patents that are cited as prior art in those patents.

The purpose of the fluorinated analog illustrated in FIG. 1 is to facilitate various types of binding, tracing, toxicology, and other studies of dextromethorphan, within the brains and bodies of humans, and of non-human animals. In particular, such analogs can be visualized by several types of non-invasive and non-destructive in vivo imaging systems, such as CAT scans, PET scans, MRI scans, and possibly even “fluoroscopic” imaging (which does not, however, relate to fluorine chemistry, and instead involves a form of live-image real-time video images comparable to moving X-ray pictures). It is hoped and believed that these types of studies will soon begin to reveal more about various relevant factors, such as the sites, concentrations, and relative binding activities of various receptor types, both in the brains and brainstems of unimpaired control subjects, and in the brains and brainstems of patients with mental impairments who are receiving substantial benefits from DM/quinidine treatment. An example of a comparable compound that enables similar research is a fluorinated analog of L-dopa, described in articles such as Endres et al 2004 and Whone et al 2004.

Accordingly, these types of studies and research can and should be advanced and accelerated, by the discoveries and disclosures herein. However, it must be recognized that these types of studies do not need to be completed, before the use of this new breakthrough can commence, in a practical and beneficial manner.

In particular, it is hoped and anticipated that this new form of treatment may be able to offer new and highly useful alternatives, for treating patients who are suffering from problems that may fall into any of the following categories:

1. autism, and various milder manifestations that point and lean in that direction but that do not cross a boundary zone that would lead to a medical diagnosis that most parents dread and would strongly prefer to avoid if possible;

2. various other types of learning disorders, including, for example, attention deficit and hyperactivity disorder (commonly abbreviated as ADHD), dyslexia, and comparable problems (which may be manifested as borderline or mild retardation in some cases, and which are sometimes referred to by parents and/or teachers as “slow learning” syndromes, to avoid potentially discouraging and stigmatizing labels);

3. various types of mental turmoil (including hormone-induced turmoil) that sometimes rise to the level of serious and disruptive afflictions (sometimes leading to suicide, criminal acts, serious drug abuse, etc.) during the development and socialization of children as they progress through childhood, puberty, and adolescence;

4. various types of afflictions that, today, are most commonly treated by tranquilizers, anxiolytic drugs, pain-killers, or “self-medication” using alcohol, marijuana, cocaine, or other illicit drugs;

5. neurological disorders that are manifested in ways that are analogous to unwanted static, noise, and distractions, or that suggest a malfunctioning control system, such as nervous tics, Tourette-type actions, obsessive and/or compulsive actions or patterns, stuttering or stammering, phobias, inordinate fear of public speaking or other performance, bipolar disorder, and chronic depression.

In addition to the foregoing, this type of treatment can also be evaluated to determine whether it will help various categories of patients cope with or respond to various other problems that have mental, emotional, or similar factors or aspects, such as (for example) controlling or reducing excessive weight, controlling their diet or other activities if they suffer from an eating, metabolic, or similar disorder, coping with periods of unusual stress, etc.

These categories are not intended as comprehensive or exhaustive, and instead are meant to suggest a number of likely and promising areas in which to seek and test expansions and extensions of the discoveries disclosed herein.

It also must be emphasized that these proposed treatments are not intended in any way as an attempt or effort to reduce or minimize the variabilities that make humans individual, and interesting. Instead, these comments and disclosures must be read in light of the actual case studies provided in Examples 4-6. Those examples provide an early description of what may turn out to be an important discovery that can help people who are suffering from problems that lead to impaired school and/or work performance, social marginalization, and chronic unhappiness. Instead of trying to create higher levels of homogeneity and uniformity while suppressing variability, these treatments are instead intended to help people learn to more closely approach and achieve their full potential, in ways that will allow their differences and individualities to be explored and expressed constructively and productively, rather than being driven or distracted by chronic anger, resentment, and sullenness. To use the television analogy one last time, a better tuning system, in a television set, does not and will not end up deciding which channel a person will decide to watch. Instead, a better tuning system simply enables someone to watch a better picture, with less static and fewer distractions, no matter which channel he or she chooses at some particular time.

Similarly, it should also be understood that useful and beneficial effects and results that are similar to (and that in some cases may be even more potent, effective, and useful than) the effects and results of the DM/quinidine combination may be achievable, by means of other known drugs that can exert the same or similar combinations of receptor activities that are exerted by DM, when its concentration in circulating blood is extended and prolonged by a P450-2D6 enzyme inhibitor such as quinidine. For example, combinations of memantine (a relatively mild NMDA antagonist drug) with one or more drugs that stimulate activity at sigma-1 and possibly sigma-2 receptors (such as the drug candidates known as J01783, OPC-14 523, and SA4503, mentioned above and in articles such as Takebayashi et al 2004 and Volz et al 2004) may well be able to accomplish either or both of the following: (i) provide useful therapeutic results, when administered in combination to various classes of patients who are suffering from impaired motor control or mental functioning; and/or, (ii) help researchers isolate, study, and evaluate the relative importance and contributions of, the various different neuronal receptor activities that are being exerted by DM, when it is coadministered along with quinidine or another P450-2D6 oxidase inhibitor.

This invention also discloses methods for manufacturing a medicament, and a medicament that has been manufactured by this method. In order to qualify under such claims, the medicament must have been demonstrated in human clinical trials to provide both (i) improved motor control, in at least some patients who suffer from impaired motor control, and (ii) improved higher mental functioning, in at least some patients suffering from impaired higher mental functioning. The method for manufacturing the medicament comprising the following steps:

(a) preparing a drug mixture comprising dextromethorphan and at least one second drug that inhibits metabolic degradation of dextromethorphan, and,

(b) packaging the drug mixture within a package that informs physicians and prospective consumers that the drug mixture has been demonstrated to be effective in providing at least one of: (i) improved motor control, in at least some patients who suffer from impaired motor control; and, (ii) improved higher mental functioning, in at least some patients suffering from impaired higher mental functioning.

One of the limitations in the medicament claims refers to, “packaging the drug mixture within a package that informs physicians and prospective consumers that the drug mixture has been demonstrated to be effective . . . ” That limitation is intended to be fully consistent and compatible with the legal requirements enforced by the U.S. Food and Drug Administration (and similar agencies in other countries), which explicitly require a drug, and the labeling information on the package used to sell the drug, to be treated and regarded legally as a single item of commerce, which must be considered and evaluated as an integral and indivisible unit.

### EXAMPLES

### Example 1

### First Patient with Amyotrophic Lateral Sclerosis

The patient described in this first example is a male who began to have problems with his left foot in September 2000, while in his late 40's. His legs gradually weakened, leading to problems walking. He was diagnosed with amyotrophic lateral sclerosis (ALS, also known as Lou Gehrig's disease). As his disease progressed, he suffered from weakness in his arms and hands, increased saliva production, difficulty in speaking and swallowing, and fairly frequent choking. Those problems led to difficulty in eating, and he began losing weight.

He also began to experience problems controlling his emotions; several times a week, he would inexplicably become tearful. Because of this problem, he was enrolled in a clinical trial of DM/quinidine for emotionality, at a daily dosage of 30 mg each, every 12 hr, for 60 mg of each per day.

In a followup consultation, he reported that his emotionality was well controlled, and his speech was better. It remained somewhat slurred, with nasal tonality, but he could be understood without serious difficulty.

In April 2002, he reported that his speech deteriorated, after he had stopping taking the DM/quinidine combination for three weeks. Upon resuming that drug combination, his speech again improved.

He was seen again in July of 2002, while still on the medication. His speech was somewhat slurred, but easily understood. He reported that he was having less trouble handling salivary secretions, and his swallowing was improved. He still occasionally coughed after drinking liquids, but he had gained back weight he had lost, indicating that he was eating better.

In January 2003, the patient sent an e-mail to the Applicant, reporting, “Jan 08 is the one year anniversary of that drug I've been taking and it's really helping me too, swallowing and its saving my voice.”

As of April 2004, when last seen by the Applicant, this patient was still able to talk and eat without assistance, and he exhibited normal emotional responses. He elected to continue taking the DM/quinidine combination.

### Example 2

### Second Patient with ALS

The patient described in this second example is a male in his 60's, who began to experience problems with his voice (including hoarseness) in June 2002. Subsequently, his right hand became weak, and he began to have difficulty walking. He was examined and diagnosed as suffering from ALS. When first seen by the Applicant, in September 2003, he reported difficulties in swallowing, eating, and speaking, involving saliva accumulating in his mouth, food getting stuck in his throat, loss of weight, etc. He was also suffering from emotionality, and enrolled in a trial of DM/quinidine. During a follow-up examination about 3 months later, he still complained of occasional choking, but he reported that he was eating more normally, and suffering from fewer problems with swallowing. In addition, his clarity of speech was substantially improved.

When interviewed again seven months later, he reported that he was still eating normally, and he was no longer bothered by abnormal saliva secretion or accumulation. His clarity of speech also continued to remain substantially improved, compared to its pretreatment level.

When asked about his improvements in speech and swallowing, the patient commented that some of the improvement might be attributable to his use of a breathing assistance machine at night. However, the Applicant has seen those machines used many times as conventional palliative treatment for ALS patients, and in the Applicant's experience, the use of such a machine normally has no significant effects on problems involving saliva, swallowing, or speaking.

### Example 3

### Patient After Head Injury and Coma

The patient described in this example is a male in his 70's, who suffered from subdural bleeding after falling from a ladder in December 2002. He was comatose for six weeks. After emerging from the coma, he suffered from left-side weakness and difficulty speaking. As part of his rehabilitation, he was given speech therapy until May 2003. After completion of therapy, his speech was somewhat better, but still slurred and “garbled”. He also suffered from drooling, and stated that his mouth and throat always seemed to be “congested”, and that he frequently choked on food or liquids.

This patient also suffered from severe emotionality, involving an average of twenty to thirty episodes of crying each day. Because his neurologist had heard of DM/quinidine controlling emotionality in earlier trials, the patient was enrolled in an open-label trial involving the use of that drug combination, in June 2003.

Approximately one month later, in a follow-up examination, he reported (with confirmation by his wife) major improvements in his emotionality; he reported a total of only three crying episodes during an entire month of treatment, compared to twenty or more episodes per day, before the treatment.

He also reported an estimated “80%” improvement in his speech. He said that several family members, with whom he had spoken by phone, had also commented that he was speaking much more clearly, and even normally. Before the DM/quinidine treatment, it had been very hard for them to understand him on the phone; after commencing the treatment, he was again able to converse with them with little or no difficulty.

He and his wife also reported a substantial lessening of the problems he was having with saliva and swallowing. Even though he still choked occasionally when he drank liquids, he was able to eat in a substantially normal manner.

In addition, this patient also began to develop a substantially improved level of understanding and awareness of his condition, and of the roles that other people were performing. After his injury but before he began the DM/quinidine treatment, his sense of self-awareness and ego had regressed to an infant level, where he showed little or no substantial awareness of the burdens he was placing on others, each time he asked someone to do something for him. After he began the DM/quinidine treatment, he returned again to a more mature and balanced recognition and understanding of how his actions were affecting other people, and it became much easier for his spouse and other caregivers to deal with his needs without becoming angry at his lack of understanding of how his demands were affecting other people. He also began to enjoy working on crossword puzzles, which (according to his wife) would have been completely beyond his capabilities, prior to starting on the DM/quinidine regimen.

### Example 4

### First Patient with Improved Mental Functioning

The patient described in this example is a female, who was in her early 40's when first examined by the Applicant in 1998. She suffered from chronic pain related to a musculo-skeletal disorder, and she had been taking DM and morphine as a participant in a clinical trial for chronic pain. During this period, she was working in a clerical-secretarial position for a law firm. She was performing poorly, and had been told that she would be terminated unless she improved substantially, because of complaints from the attorneys about numerous problems with spelling, punctuation, and other errors that were creating serious legal risks in her work output. Unknown to them, she was working overtime, trying to keep up. Her participation in the DM/morphine trial was also inconsistent and unreliable, and at one point the pharmaceutical company sponsoring that trial considered dropping her from the program.

Subsequently the patient came under the care of the Applicant, who placed her on DM/quinidine (25 mg each, twice a day, 50 mg/day total for each). She also continued to take a reduced dosage of morphine.

Soon after beginning the DM/quinidine combination, she and the attorneys at the firm where she worked began noticing major improvements in her work performance. Within a few months, lawyers at that firm began asking that she be assigned to do their work, because she had become one of the fastest, most productive, most accurate people on their support staff. Along with an improvement in the quality of her work, her output also increased, and she no longer had to put in extra hours of overtime just to keep up.

In 2002, even though she was in her late 40's, she decided she should try to get a college degree. She enrolled in a junior college, hoping that if she did well, she could transfer to a university. Despite misgivings and fear, she began taking a few academic courses she previously would have avoided, because she previously had been unable to absorb that type of content. To her surprise, she received very good grades in those courses, and was able to maintain a high grade point average, allowing her to transfer to a university.

Reflecting on these changes in her life, she is extraordinarily grateful, and she believes the DM/quinidine combination somehow played a major and crucial role in correcting some unidentified neurological condition that was interfering with her ability to recognize, understand, and work with patterns and concepts. She reported that she can now achieve and sustain levels of concentration, logic, reasoning, and focus that she previously could not have reached or sustained. She also believes her memory is improved, and she receives a level of enjoyment and assurance she had never previously experienced, from being able to grasp concepts and connections, both in work and study, and in various other aspects of life, such as music. On that subject, she began to engage the Applicant in a discussion of music theory, and she commented that before she started the DM/quinidine treatment, she did not notice, recognize, appreciate, or think in terms of such factors.

In reply to other inquiries about how her mental performance has been affected, she offered the following comments:

(1) With regard to judgment, she was previously reckless with her personal finances; now, she manages her finances much better, has become “frugal”, and is less encumbered with debt.

(2) With regard to ideation, her level of organization and foresight are improved. She has a better grasp of what she must do to succeed at projects, ranging from a single day's work, to a much larger and longer time span (such as attending college). She is better able to plan ahead, prepare herself, and approach a task in a more logical, systematic, and effective way.

(3) She is convinced that her ability to express herself is substantially improved, and that change has improved her relationships and interactions with other people. She described her communication as being much more direct, and targeted at actually solving problems, whereas before, it had tended to be evasive, uncooperative, and “tangential” (an example of a conceptual word she used, which she probably had never used or understood prior to the DM/quinidine treatment). In class, she now enjoys participating in discussions, and prefers to sit near the front, compared to a strong preference in her earlier school years for sitting in the back of the room and trying to avoid calling attention to herself.

The Applicant, a neurologic specialist who has spent decades working with people who suffer from a wide variety of neurological problems (which inevitably create secondary stresses and strains on their ability to cope with the challenges of life), was directly and strongly impressed by both: (i) her ability to express herself in an articulate, clear, and cogent manner, and (ii) her sense of optimism, enthusiasm, and enjoyment, which clearly and unmistakably were supporting and enhancing her efforts to move forward, do better, and make constructive progress in her life, work, and relationships.

### Example 5

### Second Patient with Improved Mental Functioning

The patient described in this example is a female in her 50's who began to suffer from multiple sclerosis (MS) in the early 1990's. Prior to the MS, she had a long history of serious emotionality, which in her recollection dated to her 20's. She recognized that she was easily upset or angered, and described herself as being “either angry or crying” nearly all the time. At one time she was diagnosed as suffering from bi-polar disorder (commonly known as manic-depressive disorder), and was treated with lithium, which she said was “horrible”. Since she could not tolerate lithium, she was treated with a variety of antidepressants and anxiolytics over the next decades, all of which were associated with side effects that drove her to rotate between different treatments. Her typical pattern involved taking one or more types of antidepressant or anxiolytic drugs for as long as she could stand the side effects, then moving to a different drug for a while.

In October 2003, she began a clinical trial of DM/quinidine at 30 mg each, twice a day (60 mg total of each per day). A year later, in a telephone interview with the Applicant, she described the benefits as “amazing”, and said they had created a “remarkable transformation” in her life. Along with experiencing relief from her ongoing emotional problems, she began to enjoy unprecedented success in her business, which involves sales of consumer products. Her sales and income are greatly increased, she is now the author of a regular column in a newsletter on the types of products she sells. She also has been asked to help train, personnel, and to travel and lecture to audiences. She reported that she is “amazed” at those activities; in the past, she would have been incapable of organizing her time to a point of being able to do such things efficiently and with good results. She said she has also seen major improvements in her writing, speaking, and other communication skills, and she feels that she can now approach those types of tasks with a sense of purpose, organization, and focus that she previously never could have generated or sustained.

### Example 6

### Third Patient with Improved Mental Functioning

Another example that was brought to the attention of the Applicant involved a girl who was terribly injured in an automobile crash, when she was less than 10 years old. The bumper of an elevated truck smashed through the windshield of her car, and struck her directly in the skull. She suffered a compound fracture of the skull, with brain matter openly visible. After she was transported by helicopter to a hospital, her parents were advised by the surgeons to turn off the respirator, so she could die quietly and peacefully. They refused, and she was in a coma for months.

She slowly recovered, but only to a level of badly impaired mental functioning, requiring her to be placed in “special needs” classes with other children who suffering from serious disabilities. As an example, if a teacher asked a question, she occasionally raised her hand, thinking she knew the answer; however, if called on by the teacher, she often could not remember the answer or even the question. Those and other incidents often triggered major bouts of screaming, crying, and uncontrolled physical outbursts, often lasting for 20 minutes or more.

After more than 10 years of that type of behavior, her parents decided to have her enrolled in a clinical trial for emotional lability (also called pseudobulbar effect), using the DM/quinidine combination. Participation in the trial required the family to travel to a major city, to reach a doctor who was qualified to enroll patients in the trial.

The outcome greatly surpassed any expectations, and became a life-transforming event for her. Rather than merely helping her suppress the emotional outbursts that had characterized her life for more than 10 years, the DM/quinidine combination completely restructured and largely restored her ability to concentrate, focus, analyze, understand, and learn. Today, she behaves like an intelligent, respectful, and well-behaved young lady. On those occasions when she undergoes an episode of the type referred to as an “overload” by her parents, she has reached a point of being able to control them, to a level where her parents might notice, but no one else in the room would realize that something unusual is happening to her. As she describes it, the voices and other distractions that had tormented her “are silent now.”

Thus, there has been shown and described a new and useful method for using a combination of dextromethorphan, and a second drug that slows down the degradation of dextromethorphan, to help patients cope with impairments in motor control or mental functioning. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Baulieu E E, “Neurosteroids: a novel function of the brain,” Psychoneuroendocrinology 23(8): 963-87 (1998)

Church J, et al, “Interactions of dextromethorphan with the N-methyl-D-aspartate receptor-channel complex: single channel recordings,” Brain Res 666(2): 189-94 (1994)

Endres C J, et al, “Time profile of cerebral [18F]6-fluoro-L-DOPA metabolites in nonhuman primate: implications for the kinetics of therapeutic L-DOPA,” Frontiers Biosci. 9: 505-12 (2004)

Franklin PH, et al, “High affinity [3H]dextrorphan binding in rat brain is localized to a noncompetitive antagonist site of the activated N-methyl-D-aspartate receptor-cation channel,” Mol Pharmacol 41(1): 134-46 (1992)

Guitart X, et al, “Sigma receptors: biology and therapeutic potential,” Psychopharmacology (Berl) 174(3): 301-19 (2004)

Hayashi T, et al, “Sigma-1 receptor ligands: potential in the treatment of neuropsychiatric disorders,” CNS Drugs 18: 269-84 (2004)

Inaba, T., et al, “In vitro inhibition studies of two isozymes of human liver cytochrome P-450,” Drug Metabolism and Disposition 13: 443-447 (1985)

Inaba T, et al, “Quinidine: Potent inhibition of sparteine and debrisoquin oxidation in vivo,” Br. J. Clin. Pharmacol. 22: 199-200 (1986)

Klein M, et al, “High-affinity dextromethorphan and HPP binding sites in rat brain,” J Pharmacol Exp Ther 260(3): 990-9 (1992)

Maurice T, et al, “Neuroprotective and anti-amnesic potentials of sigma (sigma) receptor ligands,” Prog Neuropsychopharmacol Biol Psychiatry 21(1): 69-102 (1997)

Maurice T, et al, “Neuroactive neurosteroids as endogenous effectors for the sigmal receptor: pharmacological evidence and therapeutic opportunities,” Jpn J Pharmacol 81(2): 125-55 (1999)

Maurice T, et al, “Sigma(1) receptor antagonists represent a new strategy against cocaine addiction and toxicity,” Neurosci Biobehav Rev 26(4): 499-527 (2002)

Maurice T, “Neurosteroids and sigmal receptors, biochemical and behavioral relevance,” Pharmacopsychiatry 37 Suppl 3: S171-82 (2004)

Musacchio J M, et al, “Dextromethorphan binding sites in the guinea pig brain,” Cell Mol Neurobiol 8(2): 149-56 (1988)

Musacchio J M, et al, “Dextromethorphan and sigma ligands: common sites but diverse effects,” Life Sci 45(19): 1721-32 (1989)

Skuza G, et al, “Behavioral pharmacology of sigma-ligands,” Pharmacopsychiatry 37 Suppl 3: S183-8 (2004)

Su T P, et al, “Understanding the molecular mechanism of sigma-1 receptors: towards a hypothesis that sigma-1 receptors are intracellular amplifiers for signal transduction,” Curr Med Chem 10(20): 2073-80 (2003)

Takebayashi M, et al, “A perspective on the new mechanism of antidepressants: neuritogenesis through sigma-1 receptors,” Pharmacopsychiatry 37 Suppl 3: S208-13 (2004)

Volz H P, et al, “Clinical trials with sigma ligands,” Pharmacopsychiatry 37 Suppl 3: S214-20 (2004)

Whone A L, “A technique for standardized central analysis of 6-(18)F-fluoro-L-DOPA PET data from a multicenter study,” J Nucl Med. 45: 1135-45 (2004)

Yamamoto H, et al, “Sigma ligands indirectly modulate the NMDA receptor-ion channel complex on intact neuronal cells via sigma 1 site,” J Neurosci 15(1 Pt 2): 731-6 (1995)

Zhang Y, et al, “Dextromethorphan: Enhancing its systemic availability by way of low-dose quinidine-mediated inhibition of cytochrome P4502D6,” Clin. Pharmacol. Ther. 51: 647-655 (1992)

What is claimed is:

1. A method for improving motor control and higher mental function in a patient who suffers from Parkinson's disease, comprising the step of administering to said patient a drug combination comprising dextromethorphan and at least one second drug that inhibits metabolic degradation of dextromethorphan, at combined dosages that, when administered together, are effective in providing improved motor control for at least some patients who suffer from impaired motor control.

2. The method of claim 1 wherein the second drug is administered at a dosage that has been shown to significantly inhibit at least one type of oxidase enzyme that metabolizes dextromethorphan when said oxidase enzyme is not inhibited.

3. The method of claim 2 wherein the second drug comprises quinidine.

4. The method of claim 1 wherein the second drug inhibits P450-2D6 oxidase enzymes.

5. The method of claim 1 wherein dextromethorphan is administered to a patient at a dosage in a range of about 10 to about 100 milligrams per day.

6. The method of claim 1 wherein quinidine is administered to a patient at a dosage in a range of about 10 to about 100 milligrams per day.

7. A method for improving higher mental functioning in a patient suffering from Parkinson's disease, comprising the step of administering to said patient a drug combination comprising dextromethorphan and at least one second drug that inhibits metabolic degradation of dextromethorphan, at combined dosages that, when administered together, are effective in providing improved cognitive and analytical functioning among at least some patients who suffer from impaired mental functioning.

8. The method of claim 7 wherein the second drug is administered at a dosage that has been shown to significantly inhibit at least one type of oxidase enzyme that metabolizes dextromethorphan when said oxidase enzyme is not inhibited.

9. The method of claim 7 wherein the second drug comprises quinidine.

10. The method of claim 7 wherein the second drug inhibits P450-2D6 oxidase enzymes.

11. The method of claim 7 wherein dextromethorphan is administered to a patient at a dosage in a range of about 10 to about 100 milligrams per day.

12. The method of claim 7 wherein quinidine is administered to a patient at a dosage in a range of about 10 to about 100 milligrams per day.
